(19) United States
(12) Patent Application Publication
(10) Pub. No.: US 2006/0148741 A1
(43) Pub. Date: Jul. 6, 2006

(54) Metastasis suppressor gene on human chromosome 8 and its use in the diagnosis, prognosis and treatment of cancer

(75) Inventors: J. Carl Barrett, Bethesda, MD (US); Naoki Nihei, Kawachi-gun (JP); Natalay Kouprina, Potomac, MD (US); Vladimir Larionov, Potomac, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6780, US

(73) Assignee: GOVERNMENT OF THE USA, REPRESENTED BY THE SECRETARY, DEPARTMENT OF HEALTH AND HUMAN SERVICES, Rockville, MD (US)

(21) Appl. No.: 11/190,027

(22) Filed: Jul. 26, 2005

Related U.S. Application Data
(60) Provisional application No. 60/591,028, filed on Jul. 26, 2004.

(51) Int. Cl. A61K 48/00; C12Q 1/68; C07H 21/02

(52) U.S. Cl. 514/44; 435/6; 536/23.1

(57) ABSTRACT

The invention provides an isolated or purified ribonucleic acid (RNA) molecule comprising a nucleotide sequence encoded by a human (Tey1) metastasis suppressor gene located at p21-p12 on chromosome 8 or a fragment thereof, wherein the isolated or purified RNA molecule comprises from about 10 to about 100 nucleotides. The invention also provides methods of diagnosis, prognosis, and treatment of cancer, such as prostate cancer, using the isolated or purified RNA molecule.

### CROSS-REFERENCE TO RELATED APPLICATIONS

This patent application claims the benefit of U.S. Provisional Patent Application No. 60/591,028, filed Jul. 26, 2004.

### TECHNICAL FIELD OF THE INVENTION

The present invention relates to a metastasis suppressor RNA molecule encoded by the Tey1 gene located on human chromosome 8 and related vectors, host cells, compositions and methods of diagnosis, prognosis, and treatment of cancer.

### BACKGROUND OF THE INVENTION

The American Cancer Society estimates the lifetime risk that an individual will develop cancer is 1 in 2 for men and 1 in 3 for women. The development of cancer, while still not completely understood, can be enhanced as a result of a variety of risk factors. For example, exposure to environmental factors (e.g., tobacco smoke) might trigger modifications in certain genes, thereby initiating cancer development. Alternatively, these genetic modifications may not require an exposure to environmental factors to become abnormal. Indeed, certain mutations (e.g., deletions, substitutions, etc.) can be inherited from generation to generation, thereby imparting an individual with a genetic predisposition to develop cancer.

Currently, the survival rates for many cancers are on the rise. One reason for this success is improvement in the detection of cancer at a stage at which treatment can be effective. Indeed, it has been noted that one of the most effective means to survive cancer is to detect its presence as early as possible. According to the American Cancer Society, the relative survival rate for many cancers would increase by about 15% if individuals participated in regular cancer screenings. Therefore, it is becoming increasingly useful to develop novel diagnostic tools to detect the cancer either before it develops or at an as early stage of development as possible.

One popular way of detecting cancer early is to analyze the genetic makeup of an individual to detect the presence or expression levels of a marker gene(s) related to the cancer. For example, there are various diagnostic methods that analyze a certain gene or a pattern of genes to detect cancers of the breast, tongue, mouth, colon, rectum, cervix, prostate, testis, and skin.

Prostate cancer is the most common non-cutaneous malignancy diagnosed in men in the United States, accounting for over 40,000 deaths annually (see, e.g., Parker et al., J. Clin. Cancer, 46, 5 (1996)). While methods for early detection and treatment of prostate cancer have been forthcoming, there is an obvious need for improvement in this area. Therefore, the discovery of gene mutations which are good indicators of cancer, and more particularly prostate cancer, would be a tremendous step towards understanding the mechanisms underlying cancer and could offer a dramatic improvement in the ability of scientists to detect cancer and to predict an individual's susceptibility to a particular type of cancer.

Much research has, in fact, been centered on establishing a genetic link to prostate cancer and studies have identified many recurring genetic changes associated with prostate cancer. These genetic changes include DNA hypermethylation, allelic loss, aneuploidy, aneusomy, various point mutations, and changes in protein expression level (e.g., E-cadherin/alpha-catenin). Researchers have also discovered losses and duplications in particular chromosomes or chromosome arms which are associated with prostate cancer (see, e.g., U.S. Pat. No. 5,925,519 and Visakorpi, Ann. Chirur. Gynaec., 88, 11-16 (1999)). In particular, losses of chromosomes 6q, 8p, 10q, 13q and 16q, and duplications of chromosomes 7, 8q and Xq have be an associated with prostate cancer. Moreover, researchers have performed genetic epidemiological studies of affected populations and have identified various putative prostate cancer susceptibility loci, indicating that there is significant genetic heterogeneity in prostate cancer. These loci include Xq27-q28 (see, e.g., Xu et al., Nat. Genet., 20, 175-179 (1998)) and 1q42-q43 (see, e.g., Gibbs et al., Am. J. Hum. Genet., 64, 1087-1095 (1999) and Berthon et al., Am. J. Hum. Genet., 62, 1416-1424 (1998)).

One such potential prostate cancer susceptibility locus is the 1 q24-q31 locus (flanked by D1S2883 and D1S422), which has been designated as HPC1, due to its putative link to hereditary prostate cancer (HPC). This HPC1 locus was identified in a genome-wide scan of families at high risk for prostate cancer (see, e.g., Smith et al., Science, 274, 1371-1374 (1996)). The HPC1 locus has been controversial, however, due to the fact that researchers have had difficulty duplicating the results of Smith et al. (see, e.g., De la Chapelle et al., Curr. Opin. Genet. Dev., 8, 298-303 (1998)). In fact, some groups of researchers have found no linkage of the HPC1 locus to hereditary prostate cancer (see, e.g., Eeles et al., Am. J. Hum. Genet., 62, 653-658 (1998), Thibodeau et al., Am. J. Hum. Genet., 61(suppl.), 1733 (1997), and Mclndoe et al., Am. J. Hum. Genet., 61, 347-353 (1997)), while others have found linkage in a very small fraction of high-risk prostate cancer families (see, e.g., Schleutker et al., Am. J. Hum. Genet., 61(suppl.), 1711 (1997)). Further support for the linkage between the HPC1 locus and hereditary prostate cancer was revealed, however, via a combined Consortium analysis of 6 markers in the HPC1 region in 772 families segregating hereditary prostate cancer (see, e.g., Xu et al., Am. J. Hum. Genet., 66, 945-957 (2000)). Thus, research findings concerning the HPC1 locus and its potential link to prostate cancer have been promising, but often nonconforming.

There also have been numerous reports of allelic loss of the p arm of chromosome 8 associated with prostate cancers. Indeed, it has been estimated that as many as 65% of prostate carcinomas exhibit loss of the p arm of chromosome 8 (see, e.g., U.S. Pat. No. 6,043,088). Specific regions of chromosome 8 have been associated with cancer, specifically prostate cancer, and cancer metastasis (see, e.g., U.S. Pat. Nos. 5,882,864; 5,972,615; 6,156,515; 6,171,796; and 6,218,529, Ichikawa et al., Cancer Research, 54, 2299-2302 (1994), Kuramochi et al., The Prostate, 31, 14-20 (1997), Nihei et al., Genes, Chromosomes & Cancer, 17, 260-268 (1996), Ichikawa et al., Asian J. of Andrology, 2(3), 167-171 (2000), Ichikawa et al., The Prostate, Supplement 6, 31-35 (1996), Nihei et al., Proc. 90th Ann. Mtg. of the Amer. Assoc. Cancer Research, 40, 105 (Abstract No. 699) (March 1999), Sunwoo et al., Oncogene, 18, 2651-2655 (1999), Trapman et al., Cancer Research, 54, 6061-6064 (1994), Konig et al., Urol. Res., 27(1), 3-8 (1999), Kagan et al., Oncogene, 11, 2121-2126 (1995), He et al., Genomics, 43, 69-77 (1997), U.S. Pat. No. 6,043,088, Levy et al., Genes, Chromsomes & Cancer, 24, 42-47 (1999), International Patent Application No. WO 99/32644, Wang et al., Genomics, 60, 1-11 (1999), Oba et al., Cancer Genet. Cytogenet., 124, 20-26 (2001), and Suzuki et al., Genes, Chromsomes & Cancer, 13, 168-174 (1995)).

A chromosomal breakpoint at 8p11 has been found to be a recurrent chromosomal breakpoint in prostate cancer cell lines (see, e.g., Pan et al., Genes, Chromosomes & Cancer, 30, 187-195 (2001)). It also has been reported that loss of 8p sequences may result from complex structural rearrangements involving chromosome 8, which sometimes includes i(8q) chromosome formation (see, e.g., Macoska et al., Cancer Research, 55, 5390-5395 (1995) and Cancer Genet. Cytogenet., 120, 50-57 (2000)). Genetic changes at 8q in clinically organ-confined prostate cancer also have been noted (see, e.g., Fu et al., Urology, 56, 880-885 (2000)). Differential expression of the gene GC84 at 8q11 has been associated with the progression of prostate cancer (see, e.g., Chang et al., Int. J. Cancer, 83, 506-511 (1999)). 8p22 loss with 8c gain has been associated with poor outcome in prostate cancer (see, e.g., Macoska et al., Urology, 55, 776-782 (2000)), and Arbieva et al., Genome Research, 10, 244-257 (2000)). Loss of 8p23 and 8q12-13 has been found to be associated with human prostate cancer (see, e.g., Perinchery et al., Int. J. Oncology, 14, 495-500 (1999)). Gene amplification in 8q24 has been found to be associated with human prostate cancer (see, e.g., International Patent Application No. WO 96/20288). Mutations in the FEZ1 gene at 8p22 have been found to be associated with primary esophageal cancers and in a prostate cancer cell line (see, e.g., Ishii et al., PNAS USA, 96, 3928-3933 (March 1999)).

The use of various gene sequences in the diagnosis and prognosis of cancer, specifically prostate cancer, also has been reported (see, e.g., U.S. Pat. Nos. 5,861,248, 5,882,864, 5,925,519, 5,972,615, 5,994,071, 6,140,049, 6,156,515, 6,171,796, 6,218,529, and European Patent Application No. 1 048 740).

There remains a need for the identification of gene products which can be shown to have a strong association with cancer, such as prostate cancer. Such gene products would lead directly to early, sensitive, and accurate methods for detecting cancer or a predisposition to cancer in a mammal. Moreover, such methods would enable clinicians to monitor the onset and progression of cancer in an individual with greater sensitivity and accuracy, as well as the response of an individual to a particular treatment. The present invention provides such gene products, as well as related vectors, host cells, compositions and methods of use in the diagnosis, prognosis and treatment of cancer, particularly prostate cancer.

### BRIEF SUMMARY OF THE INVENTION

The invention provides an isolated or purified ribonucleic acid (RNA) molecule comprising a nucleotide sequence encoded by a human (Tey1) metastasis suppressor gene located at p21-p12 on chromosome 8 or a fragment thereof, wherein the isolated or purified RNA molecule comprises from about 10 to about 100 nucleotides.

The invention further provides a method of treating cancer prophylactically or therapeutically in a mammal. The method comprises administering to the mammal an effective amount of an isolated or purified RNA molecule comprising a nucleotide sequence encoded by a human (Tey1) metastasis suppressor gene located at p21-p12 on chromosome 8, or a fragment thereof. The isolated or purified RNA molecule comprises from about 10 to about 100 nucleotides, and is optionally in the form of (a) a vector or (b) a conjugate.

Still also provided by the invention is a method of diagnosing cancer in a mammal. The method comprises (a) obtaining a test sample from the mammal, and (b) assaying the test sample for the level of an RNA molecule comprising a nucleotide sequence encoded by a human (Tey1) metastasis suppressor gene located at p21-p12 on chromosome 8, or a fragment thereof. The isolated or purified RNA molecule comprises from about 10 to about 100 nucleotides, and a decrease in the level of the RNA molecule in the test sample as compared to the level of the RNA molecule in a control sample is diagnostic for the cancer.

The invention further provides a method of prognosticating cancer in a mammal. The method comprises (a) obtaining a test sample from the mammal, and (b) assaying the test sample for the level of an RNA molecule comprising a nucleotide sequence encoded by a human (Tey1) metastasis suppressor gene located at p21-p12 on chromosome 8, or a fragment thereof. The RNA molecule comprises from about 10 to about 100 nucleotides, and an increase in the level of the RNA molecule over time is indicative of a positive prognosis and a decrease in the level of the RNA molecule over time is indicative of a negative prognosis.

### BRIEF DESCRIPTION OF THE DRAWINGS

FIG. 1 is the nucleotide sequence (SEQ ID NO: 1) of the cDNA of Tey1, which is read 5′ to 3′ from top to bottom and left to right.

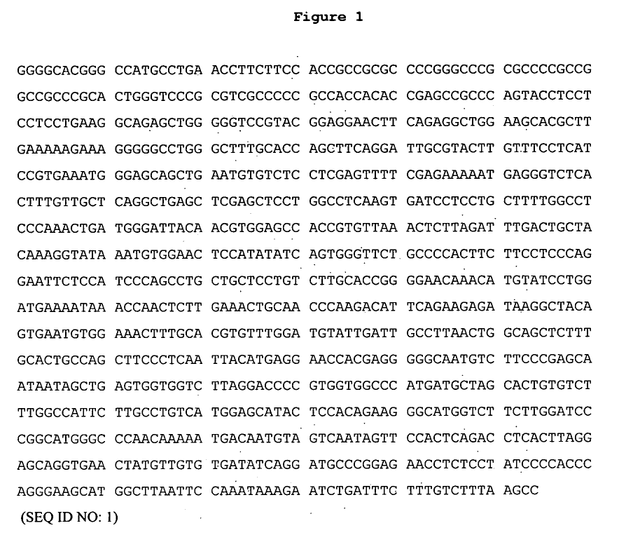

### DETAILED DESCRIPTION OF THE INVENTION

The present invention provides an isolated or purified ribonucleic acid (RNA) molecule comprising, consisting essentially of, or consisting of, a nucleotide sequence encoded by a human (Tey1) metastasis suppressor gene located at p21-p12 on chromosome 8 or a fragment thereof. The isolated or purified RNA molecule comprises from about 10 to about 100 nucleotides. The RNA may be isolated or purified from any suitable source. For example, the RNA may be isolated or purified from tissues, transcribed from an expression vector, or chemically synthesized by methods known in the art. Without desiring to be bound by any particular theory, it is believed that the inventive RNA inhibits the expression of Tey1 polypeptides, and in particular the expression of the polypeptide encoded by exon 4 of Tey1. Preferably, the RNA molecule of the invention inhibits cancer in vitro or in vivo.

An “isolated” RNA is an RNA not found in its natural environment either because of purification or because it was synthetically produced. By “purified” is meant that a given nucleic acid, whether one that has been removed from nature (including, without limitation, genomic DNA and cellular RNA) or synthesized (including cDNA) and/or amplified under laboratory conditions, has been increased in purity, wherein “purity” is a relative term, not “absolute purity.”

The terms “nucleic acid molecule,” “nucleic acid sequence,” and “nucleotide sequence” encompass a polymer of RNA or DNA, i.e., a polynucleotide, which can be single-stranded or double-stranded, and which can contain non-natural or altered nucleotides. In addition, “nucleic acid molecule,” “nucleic acid sequence,” and “nucleotide sequence” may also encompass modified nucleic acid molecules or nucleic acid derivatives, such as, for example, phosphothiorate-modified DNA or RNA, 3′-methoxy nucleic acid derivatives, and peptide nucleic acids (PNAs).

The nucleic acid molecule suppress (i.e., inhibits) tumor metastasis via suppression of expression of one or more polypeptides encoded by the Tey1 gene. Metastasis is “suppressed” when one or more tumors is prevented from spreading to a secondary site, or when one or more tumors migrates from a primary site to a secondary site but is unable to establish itself at a secondary site. Suppression of Tey1 polypeptide expression can result from inhibition of Tey1 transcription or translation, or from Tey1 protein degradation or instability post-translation.

The nucleic acid molecule can be any suitable RNA molecule, but preferably is an RNA molecule that silences expression of one or more polypeptides encoded by the Tey1 gene. Suitable RNA molecules include, for example, messenger RNA (mRNA), double-stranded RNA, small interfering RNA (siRNA), antisense RNA, retroviral insertion vectors, or any other suitable RNA molecule capable of silencing Tey1 protein expression. The RNA molecule can be complementary to the coding or non-coding strand of the Tey1 gene. The Tey1 gene preferably comprises the nucleic acid sequence of SEQ ID NO: 1. Preferably, the RNA molecule is a small, non-coding RNA molecule (i.e., a “small RNA molecule”). Small RNA molecules typically contain between 20 and 30 nucleotides, and inhibit expression of target genes through homologous sequence interactions (see, e.g., Finnegan et al., J. Cell. Sci., 116, 4689-4693 (2003)). Small RNAs, such as microRNAs, are generated via processing of longer double-stranded RNA (dsRNA) precursors by an RNAseIII-like enzyme called Dicer ribonuclease (see, e.g., Bernstein et al., Nature, 409, 363-366 (2001)). After processing by Dicer, small RNAs typically are incorporated into a ribonucleoprotein complex (see, e.g., Zeng et al., Proc. Natl. Acad. Sci. USA, 100, 9779-9784 (2003)). Most preferably, the RNA molecule is a microRNA molecule (miRNA). MicroRNAs are encoded in intergenic regions within the host genome as one arm of an approximately 70-nucleotide RNA stem-loop structure called a pre-miRNA (see, e.g., Zeng et al., supra, Lee and Ambros, Science, 294, 862-864 (2001), and Lagos-Quintana et al., Science, 294, 853-858 (2001)). Processing of miRNAs also is dependent upon the Dicer ribonuclease, and processed miRNAs are incorporated into a ribonucleoprotein complex (see, e.g., Zeng et al., supra). MicroRNAs likely silence gene expression by either base pairing with the 3′ untranslated region (UTR) of mRNA molecules to block their translation, or inducing mRNA degradation (Finnegan et al., supra).

In one embodiment, the nucleic acid sequence can encode a functional fragment of the inventive RNA molecule, i.e., any portion of the RNA molecule that retains the biological activity of the full-length RNA molecule at measurable levels. A functional RNA fragment produced by expression of the nucleic acid sequence can be identified using standard molecular biology and cell culture techniques, such as assaying the biological activity of the fragment in human cancer cells transiently transfected with a nucleic acid sequence encoding the RNA fragment.

The RNA molecule can be any suitable size, so long as the RNA molecule exhibits metastasis suppressing functions (e.g., inhibits the expression of Tey1 polypeptides). Preferably, the RNA molecule is about 500 nucleotides or fewer (e.g., about 400 nucleotides, about 200 nucleotides, or about 100 nucleotides) in length. More preferably, the RNA molecule is from about 10 nucleotides to about 200 nucleotides (e.g., about 50 nucleotides, about 100 nucleotides, or about 150 nucleotides). Most preferably, the RNA molecule is from about 10 nucleotides to about 100 nucleotides (e.g., about 10 nucleotides, about 30 nucleotides, or about 90 nucleotides) in length.

The invention further provides one or more isolated or purified deoxyribonucleic acids (DNA) molecule comprising, consisting essentially of, or consisting of, a nucleotide sequence encoding the above-described RNA molecule. The DNA molecule can comprise a nucleotide sequence that is substantially identical to the transcribed portion of the Tey1 gene, or can be one that hybridizes under low stringency conditions to an isolated or purified nucleic acid molecule comprising, consisting essentially of, or consisting of, the nucleotide sequence encoding the above-described RNA molecule, or shares 50% or more (e.g., 55%, 60%, 65%, 70%, 75% or 80% or more) identity with the DNA molecule comprising, consisting essentially of, or consisting of, a nucleotide sequence encoding the above-described RNA molecule.

Also provided is an isolated or purified DNA molecule comprising, consisting essentially of, or consisting of, a nucleotide sequence encoding a variant of a ribonucleic acid (RNA) molecule comprising, consisting essentially of, or consisting of, a nucleotide sequence encoded by a human (Tey1) metastasis suppressor gene located at p21-p12 on chromosome 8 or a fragment thereof. The variant comprises one or more insertions, deletions, substitutions, and/or inversions as compared to the corresponding unmodified RNA molecule. Desirably, the variant Tey1 RNA molecule does not differ functionally from the corresponding unmodified Tey1 RNA molecule, such as that described above (e.g., inhibits the expression of a Tey1 polypeptide). Preferably, the variant RNA molecule is able to suppress metastasis of a highly metastatic prostatic tumor cell line in vivo at least about 75% (e.g., about 75%, about 80%, or about 85%), more preferably at least about 90% (e.g., about 90%, about 95%, or about 100%), as well as the unmodified RNA molecule as determined by an in vivo assay. The manner in which the assay is carried out is not critical and can be conducted in accordance with methods known in the art. Preferably, the assay is carried out in accordance with the Example set forth herein.

The present invention also provides an isolated or purified DNA molecule comprising, consisting essentially of, or consisting of, a nucleotide sequence that is complementary to a DNA molecule encoding an RNA molecule comprising, consisting essentially of, or consisting of, a nucleotide sequence encoded by a human (Tey1) metastasis suppressor gene located at p21-p12 on chromosome 8, or a fragment thereof. In this respect, the complementary DNA molecule preferably hybridizes under low stringency, or preferably high stringency, conditions to an isolated or purified DNA molecule encoding the inventive RNA molecule, or a fragment thereof, or shares 50% or more (e.g., 55%, 60%, 65%, 70%, 75% or 80% or more) identity with the DNA molecule that encodes the inventive RNA molecule. High stringency and low stringency conditions are defined below.

Also provided is an isolated or purified DNA molecule comprising, consisting essentially of, or consisting of, a nucleotide sequence that is complementary to a DNA molecule encoding a variant Tey1 RNA molecule as described above.

With respect to the above, one of ordinary skill in the art knows how to generate insertions, deletions, substitutions and/or inversions in a given nucleic acid molecule (see, e.g., Sambrook et al., Molecular Cloning, a Laboratory Manual, 3rd edition, Cold Spring Harbor Press, Cold Spring Harbor, N.Y. (2001), and Ausubel et al., Current Protocols in Molecular Biology, Greene Publishing Associates and John Wiley & Sons, New York, N.Y. (1994)). With respect to the above isolated or purified nucleic acid molecules, it is preferred that any such insertions, deletions, substitutions and/or inversions are introduced such that the metastasis suppressor activity is not compromised, or is enhanced.

Alterations of the nucleotide sequence of the inventive RNA molecule to produce variant RNA molecules can be made by a variety of means known to those skilled in the art. For instance, site-specific mutations in the DNA molecule encoding the inventive RNA molecule can be introduced by ligating into an expression vector a synthesized oligonucleotide comprising the modified site. In another alternative, oligonucleotide-directed site-specific mutagenesis can be performed using the methods disclosed in, for example, Walder et al., Gene, 42, 133-139 (1986), Bauer et al., Gene, 37, 73-81 (1985), Craik, Biotechniques, 3, 12-19 (1995), and U.S. Pat. Nos. 4,518,584 and 4,737,462. Similarly, techniques employing chemical synthesis can be used.

A variant Tey1 RNA molecule does not differ functionally from the unmodified Tey1 RNA molecule if it can suppress metastasis of a tumor, particularly a prostatic tumor. This metastasis suppressing activity can be tested via any suitable assay. Preferred assays include those employing analysis of the in vivo metastasis suppression of AT6.3 cells, such as the assay described herein in the Example. A variant RNA molecule suitable in the context of the present invention can be more or less active than the unmodified Tey1 RNA molecule.

An indication that polynucleotide sequences are substantially identical is if two molecules selectively hybridize to each other under stringent conditions. The phrase “hybridizes to” refers to the selective binding of a single-stranded nucleic acid probe to a single-stranded target DNA or RNA sequence of complementary sequence when the target sequence is present in a preparation of heterogeneous DNA and/or RNA. “Stringent conditions” are sequence-dependent and differ according to the particular circumstances. Generally, stringent conditions are selected to be about 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe.

For example, under stringent conditions, as that term is understood by one skilled in the art, hybridization is preferably carried out using a standard hybridization buffer at a temperature ranging from about 50° C. to about 75° C., even more preferably from about 60° C. to about 70° C., and optimally from about 65° C. to about 68° C. Alternately, formamide can be included in the hybridization reaction, and the temperature of hybridization can be reduced to preferably from about 35° C. to about 45° C., even more preferably from about 40° C. to about 45° C., and optimally to about 42° C. Desirably, formamide is included in the hybridization reaction at a concentration of from about 30% to about 50%, preferably from about 35% to about 45%, and optimally at about 40%. Moreover, optionally, the hybridized sequences are washed (if necessary to reduce non-specific binding) under relatively highly stringent conditions, as that term is understood by those skilled in the art. For instance, desirably, the hybridized sequences are washed one or more times using a solution comprising salt and detergent, preferably at a temperature of from about 50° C. to about 75° C., even more preferably at from about 60° C. to about 70° C., and optimally from about 65° C. to about 68° C. Preferably, a salt (e.g., such as sodium chloride) is included in the wash solution at a concentration of from about 0.01 M to about 1.0 M. Optimally, a detergent (e.g., such as sodium dodecyl sulfate) is also included at a concentration of from about 0.01% to about 1.0%. The following is an example of highly stringent conditions for a Southern hybridization in aqueous buffers (no formamide) (see, e.g., Sambrook et al., supra):

Hybridization Conditions:

6×SSC or 6×SSPE

5× Denhardt's Reagent

1% SDS

100 ug/ml salmon sperm DNA

hybridization at 65-68° C.

Washing Conditions:

0.1×SSC/0.1% SDS

washing at 65-68° C.

Exemplary moderately stringent conditions, which allow for about 25% mismatch, are as follows:

Hybridization Conditions:

5×SSC or 5×SSPE

5× Denhardt's Reagent

100 μg/ml salmon sperm DNA

hybridization at 50° C.

Washing Conditions:

1×SSC/0.1% SDS

washing at 55° C.

Exemplary low stringency conditions, which allow for about 50% mismatch, are as follows:

Hybridization Conditions:

5×SSC or 5×SSPE

5× Denhardt's Reagent

100 μg/ml salmon sperm DNA hybridization at 25° C.

Washing Conditions:

2×SSC/0.1% SDS

washing at 37° C.

In view of the above, “highly stringent conditions” allow for up to about 20% mismatch, preferably up to about 15% mismatch, more preferably up to about 10% mismatch, and most preferably less than about 5% mismatch, such as 4%, 3%, 2% or 1% mismatch. “At least moderately stringent conditions” preferably allow for up to about 45% mismatch, more preferably up to about 35% mismatch, and most preferably up to about 25% mismatch. “Low stringency conditions” preferably allow for up to 60% mismatch, more preferably up to about 50% mismatch. With respect to the preceding ranges of mismatch, 1% mismatch corresponds to one degree decrease in the melting temperature.

The above isolated or purified nucleic acid molecules also can be characterized in terms of “percentage of sequence identity.” In this regard, a given nucleic acid molecule as described above can be compared to a nucleic acid molecule encoding a corresponding gene (i.e., the reference sequence) by optimally aligning the nucleic acid sequences over a comparison window, wherein the portion of the polynucleotide sequence in the comparison window may comprise additions or deletions (i.e., gaps) as compared to the reference sequence, which does not comprise additions or deletions, for optimal alignment of the two sequences. The percentage of sequence identity is calculated by determining the number of positions at which the identical nucleic acid base occurs in both sequences, i.e., the number of matched positions, dividing the number of matched positions by the total number of positions in the window of comparison, and multiplying the result by 100 to yield the percentage of sequence identity. Optimal alignment of sequences for comparison may be conducted by computerized implementations of known algorithms (e.g., GAP, BESTFIT, FASTA, and TFASTA in the Wisconsin Genetics Software Package, Genetics Computer Group (GCG), 575 Science Dr., Madison, Wis., or BlastN and BlastX available from the National Center for Biotechnology Information, Bethesda, Md.), or by inspection. Percent sequence identity can be determined using BESTFIT or BlastN with default parameters.

“Significant sequence identity” means that preferably at least 45%, more preferably at least 50%, and most preferably at least 55% (such as 60%, 65%, 70%, 75%, 80%, 85%, 90%, 95% or more) of the sequence of a given nucleic acid molecule is identical to a given reference sequence. Typically, two polypeptides are considered to have “substantial sequence identity” if at least 45%, preferably at least 60%, more preferably at least 90%, and most preferably at least 95% (e.g., 96%, 97%, 98% or 99%) of the amino acids of which the polypeptides are comprised are identical to or represent conservative substitutions of the amino acids of a given reference sequence.

The above-described nucleic acid molecules can be used, in whole or in part (i.e., as fragments or primers), to identify and isolate related genes from humans (and other mammals) for use in the context of the present inventive methods using conventional means known in the art (see, e.g., Birren et al., Genome Analysis: A Laboratory Manual Series, Volume 1, Analyzing DNA, Cold Spring Harbor Laboratory Press, Cold Spring Harbor, N.Y. (1997), Birren et al., Genome Analysis: A Laboratory Manual Series, Volume 2, Detecting Genes, Cold Spring Harbor Laboratory Press, Cold Spring Harbor, N.Y. (1998), Birren et al., Genome Analysis: A Laboratory Manual Series, Volume 3, Cloning Systems, Cold Spring Harbor Laboratory Press, Cold Spring Harbor, N.Y. (1999), and Birren et al., Genome Analysis: A Laboratory Manual Series, Volume 4, Mapping Genomes, Cold Spring Harbor Laboratory Press, Cold Spring Harbor, N.Y. (1999)).

The present invention also provides a vector comprising an above-described isolated or purified nucleic acid molecule. A nucleic acid molecule as described above can be cloned into any suitable vector and can be used to transform or transfect any suitable cell or host (including, without limitation, non-human hosts). The selection of vectors and methods to construct them are commonly known to persons of ordinary skill in the art and are described in general technical references (see, in general, “Recombinant DNA Part D,” Methods in Enzymology, 153, Wu and Grossman, eds., Academic Press (1987), Sambrook et al., supra, and Ausubel et al., supra). Desirably, the vector comprises regulatory sequences, such as transcription and translation initiation and termination codons, which are specific to the type of host (e.g., bacterium, fungus, plant or animal) into which the vector is to be introduced, as appropriate and taking into consideration whether the vector is DNA or RNA. Preferably, the vector comprises regulatory sequences that are specific to the genus of the host. Most preferably, the vector comprises regulatory sequences that are specific to the species of the host.

Constructs of vectors, which are circular or linear, can be prepared to contain an entire nucleic acid sequence as described above or a portion thereof ligated to a replication system functional in a prokaryotic or eukaryotic host cell. Replication systems can be derived from ColE1, 2 mp plasmid, λ, SV40, bovine papilloma virus, and the like.

In addition to the replication system and the inserted nucleic acid, the construct can include one or more marker genes, which allow for selection of transformed or transfected hosts. Marker genes include biocide resistance, e.g., resistance to antibiotics, heavy metals, etc., complementation in an auxotrophic host to provide prototrophy, and the like.

Suitable vectors include those designed for propagation and expansion or for expression or both. A preferred cloning vector is selected from the group consisting of the pUC series, the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as kGT10, kGT11, XZapII (Stratagene), λ EMBL4, and λ NM1149, also can be used. Examples of plant expression vectors include pBI101, pBI101.2, pBI101.3, pBI121 and pBIN19 (Clontech). Examples of animal expression vectors include pEUK-C₁, pMAM and pMAMneo (Clontech).

An expression vector can comprise a native or normative promoter operably linked to an isolated or purified nucleic acid molecule as described above. The selection of promoters, e.g., strong, weak, inducible, tissue-specific, and developmental-specific, is within the skill in the art. Similarly, the combining of a nucleic acid molecule as described above with a promoter is also within the skill in the art (see, e.g., Sambrook et al., supra, and Ausubel et al., supra).

The invention also provides a host cell comprising and expressing an isolated or purified nucleic acid molecule, optionally in the form of a vector, as described above. Examples of host cells include, but are not limited to, a human cell, a human cell line, Escherichia coli cell lines (e.g., E. coli TB-1, TG-2, DH5a, XL-Blue MRF′ (Stratagene), SA2821, and Y1090), B. subtilis, P. aerugenosa, S. cerevisiae, N. crassa, mammalian or insect host cell systems (e.g., Sf9, Ea4) including baculovirus systems (e.g., as described by Luckow, Curr. Opin. Biotechnol., 4(5), 564-72 (1993)), and established cell lines such as the COS-7, C127, 3T3, CHO, HeLa, BHK cell line, and the like, and others set forth herein below.

The present invention also provides a conjugate comprising an above-described isolated, or purified RNA or DNA molecule or fragment thereof, and a therapeutically or prophylactically active agent. “Prophylactically” as used herein does not necessarily mean prevention, although prevention is encompassed by the term. Prophylactic activity also can include lesser effects, such as inhibition of the spread of cancer. Preferably, the active agent is an anti-cancer agent. The anti-cancer agent can be a chemotherapeutic agent, e.g., a polyamine or an analogue thereof. Examples of therapeutic polyamines include those set forth in U.S. Pat. Nos. 5,880,161, 5,541,230 and 5,962,533, Saab et al., J. Med. Chem., 36, 2998-3004 (1993), Bergeron et al., J. Med. Chem., 37(21), 3464-3476 (1994), Casero et al., Cancer Chemother. Pharmacol., 36, 69-74 (1995), Bemacki et al., Clin. Cancer Res., 1, 847-857 (1995)); Bergeron et al., J. Med. Chem., 40, 1475-1494 (1997); Gabrielson et al., Clinical Cancer Res., 5, 1638-1641 (1999), and Bergeron et al., J. Med. Chem., 43, 224-235 (2000), which can be administered alone or in combination with other active agents, such as cis-diaminedichloroplatinum (II) and 1,3-bis(2-chloroethyl)-1-nitrosourea.

Methods of RNA and DNA conjugation are known in the art. In addition, conjugate kits are commercially available. Methods of conjugation and conjugates are described in, for example, Hermanson, G. T., Bioconjugate Techniques, Academic Press, San Diego, Calif. (1996), and Muratovska, FEBS Lett., 558(1-3), 63-8 (2004), and U.S. Pat. Nos. 6,013,779, 6,274,552, and 6,080,725.

The present invention also provides a composition comprising an above-described isolated or purified RNA or DNA molecule, optionally in the form of a vector or a conjugate comprising a prophylactically or therapeutically active agent, and pharmaceutically acceptable carrier. Pharmaceutically acceptable carriers are well-known in the art, and are readily available. The choice of carrier will be determined in part by the particular route of administration and whether a nucleic acid molecule or a polypeptide molecule (or conjugate thereof) is being administered. Accordingly, there is a wide variety of suitable formulations for use in the context of the present invention, and the invention expressly provides a pharmaceutical composition that comprises an active agent of the invention and a pharmaceutically acceptable excipient/adjuvant. The following methods and excipients/adjuvants are merely exemplary and are in no way limiting.

Formulations suitable for oral administration can consist of (a) liquid solutions, such as an effective amount of the compound dissolved in diluent, such as water, saline, or orange juice; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth. Pastilles can comprise the active ingredient in an inert base, such as gelatin and glycerin, or sucrose and acacia, emulsions, gels, and the like containing, in addition to the active ingredient, such excipients/carriers as are known in the art.

An active agent of the present invention, either alone or in combination with other suitable components, can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured preparations such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Additionally, active agents of the present invention can be made into suppositories by mixing with a variety of bases such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate. Further suitable formulations are found in Remington's Pharmaceutical Sciences, 17th ed., Mack Publishing Company, Philadelphia, Pa. (1985), and methods of drug delivery are reviewed in, for example, Langer, Science, 249, 1527-1533 (1990).

In addition, the composition can comprise additional therapeutic or biologically-active agents. For example, therapeutic factors useful in the treatment of a particular indication can be present. Factors that control inflammation, such as ibuprofen or steroids, can be part of the pharmaceutical composition to reduce swelling and inflammation associated with in vivo administration of the RNA or DNA molecule and physiological distress. Immune enhancers can be included in the pharmaceutical composition to upregulate the body's natural defenses against disease. Moreover, cytokines can be administered with the composition to attract immune effector cells to the disease (e.g., tumor) site.

Anti-angiogenic factors, such as soluble growth factor receptors (e.g., sflt), growth factor antagonists, and the like, also can be part of the composition. Similarly, vitamins and minerals, anti-oxidants, and micronutrients can be co-administered with the composition. Antibiotics, i.e., microbicides and fungicides, can be present to reduce the risk of infection associated with gene transfer procedures and other disorders. The addition of chemotherapeutic agents to the pharmaceutical composition can provide an additional mechanism of effecting tumor reduction.

The invention further provides a method of treating cancer prophylactically or therapeutically in a mammal. The method comprises administering to the mammal at risk, or in need of prophylaxis or therapy, an effective amount of the inventive RNA or DNA molecule. Preferably, the method comprises an isolated or purified RNA molecule comprising a nucleotide sequence encoded by a human (Tey1) metastasis suppressor gene located at p21-p12 on chromosome 8, or a fragment thereof, wherein the isolated or purified RNA molecule comprises from about 10 to about 100 nucleotides, optionally in the form of (a) a vector or (b) a conjugate, whereupon the mammal is treated for the cancer prophylactically or therapeutically. The method can be used to treat any type of cancer. In this regard, the cancer can be located in the oral cavity and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid) and is not necessarily a primary tumor. Tissues associated with the oral cavity include, but are not limited to, the tongue and tissues of the mouth. The cancer can arise in tissues of the digestive system including, for example, the esophagus, stomach, small intestine, colon, rectum, anus, liver, gall bladder, and pancreas. Cancers of the respiratory system can affect the larynx, lung, and bronchus and include, for example, non-small cell lung carcinoma. The cancer can arise in the uterine cervix, uterine corpus, ovary vulva, vagina, prostate, testis, and penis, which make up the male and female genital systems, and the urinary bladder, kidney, renal pelvis, and ureter, which comprise the urinary system. The cancer also can be a lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, and the like). Preferably, the cancer is prostate cancer.

The RNA or DNA molecule can be administered to the mammal using any suitable method, including intratumoral and peritumoral routes of administration. Alternatively, the RNA or DNA molecule can be administered to the mammal by targeting the RNA or DNA molecule to tumor or cancer cells. In this regard, the RNA or DNA molecule can be conjugated to a targeting moiety that selectively binds to cancer-specific cell surface molecules or antigens, thereby facilitating delivery of the RNA or DNA molecule to cancer cells. In this regard, any suitable molecule that can be linked with the therapeutic RNA or DNA molecule directly or indirectly, such as through a suitable delivery vehicle, such that the targeting moiety binds to a cell-surface receptor, can be used. The targeting moiety can bind to a cell through a receptor, a substrate, an antigenic determinant or another binding site on the surface of the cell. Examples of a targeting moiety include an antibody (i.e., a polyclonal or a monoclonal antibody), an immunologically reactive fragment of an antibody, an engineered immunoprotein and the like, a protein (e.g., where target is receptor, as substrate, or regulatory site on DNA or RNA), a polypeptide (e.g. where target is receptor), a peptide (e.g., where target is receptor), a nucleic acid, which is DNA or RNA (i.e., single-stranded or double-stranded, synthetic or isolated and purified from nature; target is complementary nucleic acid), a steroid (e.g., where target is steroid receptor), and the like. Analogs of targeting moieties that retain the ability to bind to a defined target also can be used. In addition, synthetic targeting moieties can be designed. Alternatively, the RNA molecule can be encapsulated in a liposome comprising on its surface the targeting moiety.

The targeting moiety includes any linking group that can be used to join a targeting moiety to, in the context of the present invention, an above-described nucleic acid molecule. It will be evident to one skilled in the art that a variety of linking groups, including bifunctional reagents, can be used. The targeting moiety can be linked to the therapeutic nucleic acid by covalent or non-covalent bonding. If bonding is non-covalent, the conjugation can be through hydrogen bonding, ionic bonding, hydrophobic or van der Waals interactions, or any other appropriate type of binding.

Examples of cancer-specific, cell-surface molecules include placental alkaline phosphatase (testicular and ovarian cancer), pan carcinoma (small cell lung cancer), polymorphic epithelial mucin (ovarian cancer), prostate-specific membrane antigen, α-fetoprotein, B-lymphocyte surface antigen (B-cell lymphoma), truncated EGFR (gliomas), idiotypes (B-cell lymphoma), gp95/gp97 (melanoma), N-CAM (small cell lung carcinoma), cluster w4 (small cell lung carcinoma), cluster 5A (small cell carcinoma), cluster 6 (small cell lung carcinoma), PLAP (seminomas, ovarian cancer, and non-small cell lung cancer), CA-125 (lung and ovarian cancers), ESA (carcinoma), CD19, 22 or 37 (B-cell lymphoma), 250 kD proteoglycan (melanoma), P55 (breast cancer), TCR-IgH fusion (childhood T-cell leukemia), blood group A antigen in B or 0 type individual (gastric and colon tumors), erbB-2, erbB-3, erbB-4, IL-2 (lymphoma and leukemia), IL-4 (lymphoma and leukemia), IL-6 (lymphoma and leukemia), MSH (melanoma), transferrin (gliomas), tumor vasculature integrins and the like (see, e.g., U.S. Pat. No. 6,080,725). Preferred cancer-specific, cell-surface molecules include erbB-2 and tumor vasculature integrins, such as CD11a, CD11b, CD11c, CD18, CD29, CD51, CD61, CD66d, CD66e, CD106, and CDw145.

As discussed above, to target the RNA or DNA molecule to cancer cells, the RNA or DNA molecule can be conjugated to an antibody that binds a cancer-specific cell surface molecule or antigen. A number of such antibodies are known in the art, and include the carcino-embryonic antigen-specific antibodies C46 (Amersham) and 85A12 (Unipath), the placental alkaline phosphatase-specific H17E2 antibody (ICRF), the pan carcinoma-specific NR-LU-10 antibody (NeoRx Corp.), the polymorphic epithelial mucin-specific HMFC1 antibody (ICRF), the B-human chorionic gonadotropin-specific W14 antibody, the B-lymphocyte surface antigen-specific RFB4 antibody (Royal Free Hospital), the human colon carcinoma A33 antibody (Genex), the human melanoma-specific TA-99 antibody (Genex), antibodies to c-erbB2 (see, e.g., Japanese Patent Application Nos. JP 7309780, JP 8176200 and JP 7059588), and the like. ScAbs can be developed, based on such antibodies, using techniques known in the art (see, e.g., Bind et al., Science, 242, 423-426 (1988)).

Generally, when the RNA or DNA molecule (or a conjugate thereof) is administered to an animal, such as a mammal, in particular a human, it is desirable that the RNA or DNA molecule be administered in a dose of from about 1 to about 1,000 μg/kg body weight/treatment when given parenterally. Higher or lower doses may be chosen in appropriate circumstances. For instance, the actual dose and schedule can vary depending on whether the composition is administered in combination with other pharmaceutical compositions, or depending on interindividual differences in pharmacokinetics, drug disposition, and metabolism. One skilled in the art easily can make any necessary adjustments in accordance with the necessities of the particular situation.

Similarly, a vector (including, without limitation, naked vectors and viral vectors) encoding the inventive RNA molecule can be administered to the mammal so as to deliver the RNA to the mammal. In vivo, ex vivo, and in vitro methods of gene delivery are all suitable depending on the particular context.

Those of ordinary skill in the art can readily determine the amount of an above-described isolated and purified RNA molecule or DNA molecule to be administered to an animal, such as a mammal, in particular a human. The dosage will depend upon the particular method of administration, including any vector or promoter utilized. For purposes of considering the dose in terms of particle units (pu), also referred to as viral particles, it frequently can be assumed that there are 100 particles/pfu (e.g., 1×10¹² pfu is equivalent to 1×10¹⁴ pu). An amount of recombinant virus, recombinant DNA vector or RNA genome sufficient to achieve a tissue concentration of about 10² to about 10¹² particles per ml is preferred, especially of about 10⁶ to about 10¹⁰ particles per ml. In certain applications, multiple daily doses are preferred. Moreover, the number of doses will vary depending on the means of delivery and the particular recombinant virus, recombinant DNA vector or RNA genome administered.

The invention further provides a method of diagnosing cancer in a mammal. The method comprises (a) obtaining a test sample from the mammal, and (b) assaying the test sample for the level of an RNA molecule comprising a nucleotide sequence encoded by a human (Tey1) metastasis suppressor gene located at p21-p12 on chromosome 8, or a fragment thereof, wherein the RNA molecule comprises from about 10 to about 100 nucleotides. A decrease in the level of the RNA molecule in the test sample as compared to the level of the RNA molecule in a control sample is diagnostic for the cancer. The test sample used in conjunction with the invention can be any of those typically used in the art, including without limitation, tissue. Typically, the tissue is known to be, or suspected of being cancerous (e.g., metastatic) and is obtained by means of a biopsy. Such tissue can include bone marrow, lymph nodes, skin, and any organ that may develop cancerous cells.

A method of prognosticating cancer in a mammal is also provided. The method comprises (a) obtaining a test sample from the mammal, and (b) assaying the test sample for the level of an RNA molecule comprising a nucleotide sequence encoded by a human (Tey1) metastasis suppressor gene located at p21-p12 on chromosome 8, or a fragment thereof, wherein the RNA molecule comprises from about 10 to about 100 nucleotides. The level of the RNA molecule in the test sample can be measured by comparing the level of the RNA molecule in another test sample obtained from the mammal over time in accordance with the methods described above. An increase in the level of the RNA molecule over time is indicative of a positive prognosis, and a decrease in the level of the RNA molecule over time is indicative of a negative prognosis. The method can be used to assess the efficacy of treatment of the cancer.

Diagnostic methods for detecting the presence, absence, or quantity of a nucleic acid are well-known in the art. For example, several of assays are contemplated for use in the present inventive methods of diagnosing cancer. A number of these assays are described in Sambrook et al., supra. Microarrays, such as those described in U.S. Pat. Nos. 6,197,506 and 6,040,138, also can be used to detect and quantify the inventive RNA molecule. It will be understood that the type of assay used can depend on the type of tissue to be assayed.

As used herein, the term “increased level” can be defined as detecting the RNA molecule in a mammal at a level above that which is considered normal. For example, the level of the RNA molecule in a test sample is increased when the copy number of the gene encoding the Tey1 is greater than 1 (e.g., via gene amplification), or the RNA encoded by the Tey1 gene is detected in an amount of about 1-10,000 ng/ml.

When an RNA molecule is assayed, various assays can be used to measure the presence and/or level of nucleic acid present. For example, when only the detection of the RNA molecule is necessary to effectively diagnose the cancer, assays including PCR and microarray analysis can be used. In certain embodiments, it will be necessary to detect the quantity of the RNA molecule present. In these embodiments, it will be advantageous to use various hybridization techniques known in the art that can effectively measure the level of the RNA molecule in a test sample. Such hybridization techniques can include, for example, Northern hybridization, RNAse protection assays, in situ hybridization, capture assays, and microarray analysis.

It will be understood that, in such assays, a nucleic acid sequence that specifically binds to or associates with the RNA molecule, can be attached to a label for determining hybridization. A wide variety of appropriate labels are known in the art, including fluorescent, radioactive, and enzymatic labels as well as ligands, such as avidin/biotin, which are capable of being detected. Preferably, a fluorescent label or an enzyme tag, such as urease, alkaline phosphatase or peroxidase, is used instead of a radioactive or other environmentally undesirable label. In the case of enzyme tags, calorimetric indicator substrates are known which can be employed to provide a detection means visible to the human eye or spectrophotometrically to identify specific hybridization with complementary Tey1 nucleic acid-containing samples.

When a nucleic acid encoding the RNA molecule is amplified in the context of a diagnostic application, the nucleic acid used as a template for amplification optionally can be isolated from cells contained in the test sample, according to standard methodologies (see, e.g., Sambrook et al., supra). The nucleic acid can be genomic DNA or fractionated or whole cell RNA. Where RNA is used, it can be desirable to convert the RNA to cDNA.

In a typical amplification procedure, primers that selectively hybridize to nucleic acids corresponding to the nucleic acid sequence encoding the inventive RNA molecule are contacted with the nucleic acid under conditions that permit selective hybridization. Once hybridized, the nucleic acid-primer complex is contacted with amplification reagents usually including one or more enzymes that facilitate template-dependent nucleic acid synthesis.

Various template-dependent processes are available to amplify the RNA molecule present in a given test sample. As with the various assays, a number of these processes are described in Sambrook et al., supra. One of the best-known amplification methods is the polymerase chain reaction (PCR). Similarly, a reverse transcriptase PCR (RT-PCR) can be used when it is desired to convert RNA into cDNA. Alternative methods for reverse transcription utilize thermostable DNA polymerases and are described in WO 90/07641, for example.

Other methods for amplification include the ligase chain reaction (LCR), which is disclosed in U.S. Pat. No. 4,883,750; isothermal amplification, in which restriction endonucleases and ligases are used to achieve the amplification of target molecules that contain nucleotide 5′-[alpha-thio]-triphosphates in one strand (Walker et al., Proc. Natl. Acad. Sci. USA, 89, 392-396 (1992)); strand displacement amplification (SDA), which involves multiple rounds of strand displacement and synthesis, and repair chain reaction (RCR), which involves annealing several probes throughout a region targeted for amplification, followed by a repair reaction in which only two of the four bases are present. Target-specific sequences also can be detected using a cyclic probe reaction (CPR). In CPR, a probe having 3′ and 5′ sequences of non-specific DNA and a middle sequence of specific RNA is hybridized to DNA, which is present in a sample. Upon hybridization, the reaction is treated with RNase H, and the products of the probe are identified as distinctive products, which are released after digestion. The original template is annealed to another cycling probe and the reaction is repeated. Other amplification processes also are contemplated; however, the invention is not limited as to which method is used.

It will be understood that other diagnostic tests can be used in conjunction with the diagnostic tests described herein to enhance further the accuracy of diagnosing a mammal with a cancer. For example, a monoclonal antibody, such as the ones described in U.S. Pat. No. 4,569,788, can be used effectively in diagnosing small-cell lung cancer over non small-cell lung cancer.

The following references, to the extent that they provide exemplary procedural or other details supplementary to those set forth herein, are specifically incorporated herein by reference: Birren et al., Genome Analysis: A Laboratory Manual Series, Volume 1, Analyzing DNA, Cold Spring Harbor Laboratory Press, Cold Spring Harbor, N.Y. (1997), Birren et al., Genome Analysis: A Laboratory Manual Series, Volume 2, Detecting Genes, Cold Spring Harbor Laboratory Press, Cold Spring Harbor, N.Y. (1998), Birren et al., Genome Analysis: A Laboratory Manual Series, Volume 3, Cloning Systems, Cold Spring Harbor Laboratory Press, Cold Spring Harbor, N.Y. (1999), Birren et al., Genome Analysis: A Laboratory Manual Series, Volume 4, Mapping Genomes, Cold Spring Harbor Laboratory Press, Cold Spring Harbor, N.Y. (1999), Harlow et al., Antibodies: A Laboratory Manual, Cold Spring Harbor Laboratory Press, Cold Spring Harbor, N.Y. (1988), Harlow et al., Using Antibodies: A Laboratory Manual, Cold Spring Harbor Laboratory Press, Cold Spring Harbor, N.Y. (1999), Hoffman, Cancer and the Search for Selective Biochemical Inhibitors, CRC Press (1999), Pratt, The Anticancer Drugs, 2nd edition, Oxford University Press, NY (1994), and Sambrook et al., Molecular Cloning: A Laboratory Manual, 2nd edition, Cold Spring Harbor Laboratory Press, Cold Spring Harbor, N.Y. (1989).

The following example serves to illustrate the present invention and is not intended to limit its scope in any way.

### EXAMPLE

This example demonstrates that a Tey1 gene product suppresses metastasis in vivo.

cDNAs encoding the human Type 1, Type 2, and Type 3 Tey1 gene products were obtained as described in, for example, International Patent Application No. WO 03/060074. The Type 1 and Type 2 Tey1 cDNAs each encode a 7.9 kD putative protein that contains 77 amino acids and an SH3 binding domain. The Type 3 cDNA encodes a 4.2 kD putative protein of 41 amino acids, which also contains an SH3 binding domain.

Antibodies were raised against antigenic peptides derived from the Tey1 Type 1, Type 2, and Type 3 protein products using methods known in the art (see, e.g., Harlow et al., supra). The expression of the 7.9 kD and 4.2 kD Tey1 protein products was assayed by Western blot analysis using the Tey1-specific antibodies. In this regard, the following cell lines were tested: the 127-6 cell line, which contains the 60 Kb bacterial artificial chromosome (BAC) encoding the full-length Tey1 gene, the 1-2 cell line, which expresses the 7.9 kD Type 1 Tey1 protein, the III-14 cell line, which expresses the 4.2 kD Type III Tey1 protein, and the highly metastatic rat prostate cancer cell line AT6.3. Western blot analysis failed to detect either the 7.9 kD Type I/II Tey1 protein, or the 4.2 Type III Tey1 protein. A combination immunoprecipitation/Western blot analysis confirmed these results.

Mutations in putative ATG translation start sites of the Type I, Type II, and Type III Tey1 cDNA sequences were generated. Type I Tey1 mutants containing a 21 base pair deletion of the SH3 binding motif (SH3BD) were also generated. After stable transfectants of each mutant were established in AT6.3 cells, the transfectants were administered to mice for an in vivo metastasis assay. In this regard, 5×10⁵ cells/mouse were injected subcutaneously into 5-week old male nude mice. Mice were sacrificed five weeks after treatment, and lungs were harvested for metastasis analysis. The results of this experiment are set forth below in Table 1, and demonstrate that mutations in Tey1 translation start sites correlate with reduced lung metastasis.

TABLE 1

| | | | | |
|---|---|---|---|---|
| | | | | No. of |
| | | | Tumor | Lung |
| | | Tumorigenicity | Volume | Metastasis |
| Cell | Mutation Type | Per Mouse | (cm³) | Per Mouse |

Wild-type Splice Variants

| | | | | |
|---|---|---|---|---|
| AT6.3 | Parental | 5/5 | 4.7 ± 0.8 | 163 ± 18 |
| AT6.3/127-6 | BAC | 5/5 | 2.3 ± 0.3 | 40 ± 24 |
| AT6.3/Type 1-2 | Type 1 cDNA | 4/4 | 5.1 ± 1.1 | 47 ± 21 |
| AT6.3/Type 2-1 | Type 2 cDNA | 5/5 | 2.8 ± 0.2 | 13 ± 8 |
| AT6.3/Type 3-14 | Type 3 cDNA | 5/5 | 2.0 ± 0.2 | 0 |
| AT6.3/Type 1R-2 | Reverse Type 1 | 5/5 | 4.5 ± 1.1 | 155 ± 17 |
| AT6.3/Type 2R-3 | Reverse Type 2 | 5/5 | 2.2 ± 0.7 | 177 ± 44 |
| AT6.3/Type 3R-1 | Reverse Type 3 | 5/5 | 4.2 ± 0.3 | 184 ± 53 |

ATG Mutants

| | | | | |
|---|---|---|---|---|
| AT6.3/Type 1/ATT-10 | ATG-disrupted Type 1 | 5/5 | 2.2 ± 0.3 | 1 ± 1 |
| AT6.3/Type 1/ATT-14 | ATG-disrupted Type 1 | 5/5 | 2.1 ± 0.4 | 0 |
| AT6.3/Type 2/ATT-5 | ATG-disrupted Type 2 | 3/3 | 3.1 ± 0.6 | 2 ± 1 |
| AT6.3/Type 2/ATT-14 | ATG-disrupted Type 2 | 5/5 | 2.4 ± 0.7 | 0 |
| AT6.3/Type 3/ATT-1 | ATG-disrupted Type 3 | 5/5 | 2.1 ± 0.3 | 0 |
| AT6.3/Type 3/ATT-4 | ATG-disrupted Type 3 | 5/5 | 2.3 ± 0.6 | 0 |

SH3 Deletions

| | | | | |
|---|---|---|---|---|
| AT6.3/Type 1/ΔSH3-1 | SH3BD-disrupted Type 1 | 5/5 | 2.0 ± 0.2 | 6 ± 4 |
| AT6.3/Type 1/ΔSH3-5 | SH3BD-disrupted Type 1 | 5/5 | 4.5 ± 0.4 | 24 ± 12 |
| AT6.3/Type 2/ΔSH3-8 | SH3BD-disrupted Type 2 | 5/5 | 5.6 ± 1.0 | 15 ± 5 |
| AT6.3/Type 2/ΔSH3-9 | SH3BD-disrupted Type 2 | 5/5 | 2.8 ± 0.4 | 34 ± 6 |
| AT6.3/Type 3/ΔSH3-1 | SH3BD-disrupted Type 3 | 5/5 | 2.1 ± 0.2 | 35 ± 21 |
| AT6.3/Type 3/ΔSH3-7 | SH3BD-disrupted Type 3 | 5/5 | 2.1 ± 0.5 | 1 ± 1 |

Exon 4 of the Tey1 nucleic acid sequence is the only conserved exon among the Type I, Type II, and Type III Tey1 variants. Thus, to determine if exon 4 plays a role in metastasis suppression, exon 4 mutants were generated and tested using the in vivo metastasis assay described above. In this regard, the exon 4 mutants contained either a mutation of the ATG translation start site of exon 4, or a two base pair insertion within the exon 4 coding sequence. Also tested was a Type III Tey1 mutant wherein the ATG translation start sites of exon 1 and exon 4 were mutated, and a wild-type copy of exon 4. Stable transfectants of each mutant were established in AT6.3 cells, and an in vivo metastasis assay was performed in nude mice as described above. The results of this assay are set forth in Table 2.

TABLE 2

| | | | | No. of |
|---|---|---|---|---|
| | | | Tumor | Lung |
| | | | Volume | Metastasis |
| Cell | Type | Tumorigenicity | (cm³) | Per Mouse |
| AT6.3 | Parental | 5/5 | 4.9 ± 1.4 | 203 ± 43 |
| AT6.3/Mock-2 | Mock | 5/5 | 4.1 ± 0.8 | 180 ± 57 |
| AT6.3/Mock-3 | Mock | 5/5 | 4.6 ± 1.0 | 164 ± 35 |
| AT6.3/Type 3/dATT-1 | Double ATG-disrupted Type 3 | 5/5 | 3.2 ± 0.5 | 4 ± 2 |
| AT6.3/Type 3/dATT-3 | Double ATG-disrupted Type 3 | 4/4 | 7.1 ± 0.8 | 0 |
| AT6.3/Type 3/dATT-8 | Double ATG-disrupted Type 3 | 5/5 | 5.5 ± 0.6 | 48 ± 18 |
| AT6.3/Type 3/dATT-9 | Double ATG-disrupted Type 3 | 5/5 | 5.3 ± 1.1 | 1 ± 1 |
| AT6.3/exon4-1 | Exon 4 | 5/5 | 5.6 ± 1.9 | 9 ± 4 |
| AT6.3/exon4-2 | Exon 4 | 5/5 | 4.9 ± 1.3 | 3 ± 3 |
| AT6.3/exon4-7 | Exon 4 | 5/5 | 4.6 ± 0.7 | 4 ± 2 |
| AT6.3/exon4-9 | Exon 4 | 5/5 | 7.4 ± 1.7 | 24 ± 9 |
| AT6.3/exon4/ATT-1 | ATG-disrupted exon 4 | 5/5 | 4.6 ± 0.8 | 45 ± 20 |
| AT6.3/exon4/ATT-9 | ATG-disrupted exon 4 | 5/5 | 6.0 ± 1.1 | 1 ± 1 |
| AT6.3/exon4/ATT-20 | ATG-disrupted exon 4 | 5/5 | 4.4 ± 0.2 | 6 ± 5 |
| AT6.3/exon4/2bins-2 | Exon 4 with 2 bp insertion | 5/5 | 3.7 ± 0.9 | 2 ± 1 |
| AT6.3/exon4/2bins-8 | Exon 4 with 2 bp insertion | 5/5 | 5.9 ± 0.5 | 28 ± 10 |
| AT6.3/exon4/2bins-17 | Exon 4 with 2 bp insertion | 5/5 | 4.4 ± 0.2 | 0 |

The results of these experiments strongly suggest that disruption of protein translation is associated with metastasis suppression by Tey1, and that the Tey1 exon 4 sequence plays an important role in metastasis suppression. The results also strongly suggest the existence of a microRNA region within the Tey1 nucleic acid sequence that functions to silence Tey1 gene expression.

All of the references cited herein, including patents, patent applications, and publications, are hereby incorporated in their entireties by reference.

While this invention has been described with an emphasis upon preferred embodiments, it will be obvious to those of ordinary skill in the art that variations of the preferred embodiments may be used and that it is intended that the invention may be practiced otherwise than as specifically described herein. Accordingly, this invention includes all modifications encompassed within the spirit and scope of the invention as defined by the following claims.

What is claimed is:

1. An isolated or purified ribonucleic acid (RNA) molecule comprising a nucleotide sequence encoded by a human (Tey1) metastasis suppressor gene located at p21-p12 on chromosome 8 or a fragment thereof, wherein the isolated or purified RNA molecule comprises from about 10 to about 100 nucleotides.

2. An isolated or purified deoxyribonucleic acid (DNA) molecule comprising a nucleotide sequence encoding the RNA molecule of claim 1.

3. An isolated and purified RNA molecule comprising a nucleotide sequence encoding a variant of the RNA molecule of claim 1, which comprises one or more insertions, deletions, substitutions, and/or inversions, wherein the variant RNA molecule does not differ functionally from the corresponding unmodified RNA molecule.

4. The isolated or purified RNA molecule of claim 3, wherein the variant RNA molecule is able to suppress metastasis of a highly metastatic prostatic tumor cell line in vivo at least about 90% as well as the unmodified RNA molecule.

5. An isolated and purified DNA molecule comprising a nucleotide sequence encoding the RNA molecule of claim 3.

6. An isolated or purified DNA molecule comprising a nucleotide sequence that is complementary to a DNA molecule encoding the RNA molecule of claim 1.

7. An isolated or purified DNA molecule comprising a nucleotide sequence that is complementary to a nucleotide sequence encoding a variant of the RNA molecule of claim 1.

8. A vector comprising the isolated or purified DNA molecule of claim 2.

9. A vector comprising the isolated or purified DNA molecule of claim 5.

10. A vector comprising the isolated or purified DNA molecule of claim 6.

11. A vector comprising the isolated or purified nucleic acid molecule of claim 7.

12. A cell comprising and expressing the isolated or purified RNA molecule of claim 1.

13. A cell comprising and expressing the isolated or purified DNA molecule of claim 2.

14. A cell comprising and expressing the isolated or purified RNA molecule of claim 3.

15. A cell comprising and expressing the isolated or purified DNA molecule of claim 5.

16. A cell comprising and expressing the isolated or purified DNA molecule of claim 6.

17. A cell comprising and expressing the isolated or purified DNA molecule of claim 7.

18. A conjugate comprising the isolated or purified RNA molecule of claim 1 and a therapeutically or prophylactically active agent.

19. The conjugate of claim 18, wherein the therapeutically or prophylactically active agent is an anti-cancer agent.

20. A conjugate comprising the isolated or purified RNA molecule of claim 3 and a therapeutically or prophylactically active agent.

21. The conjugate of claim 20, wherein the therapeutically or prophylactically active agent is an anti-cancer agent.

22. A composition comprising the isolated or purified RNA molecule of claim 1, optionally in the form of a conjugate, comprising a therapeutically or prophylactically active agent, and a pharmaceutically acceptable carrier.

23. A composition comprising the isolated or purified RNA molecule of claim 3, optionally in the form of a conjugate, comprising a therapeutically or prophylactically active agent, and a pharmaceutically acceptable carrier.

24. A method of treating cancer prophylactically or therapeutically in a mammal, which method comprises administering to the mammal an effective amount of an isolated or purified RNA molecule comprising a nucleotide sequence encoded by a human (Tey1) metastasis suppressor gene located at p21-p12 on chromosome 8, or a fragment thereof, wherein the isolated or purified RNA molecule comprises from about 10 to about 100 nucleotides, optionally in the form of (a) a vector or (b) a conjugate, whereupon the mammal is treated for the cancer prophylactically or therapeutically.

25. The method of claim 24, wherein the cancer is prostate cancer.

26. A method of diagnosing cancer in a mammal, which method comprises:
(a) obtaining a test sample from the mammal, and
(b) assaying the test sample for the level of an RNA molecule comprising a nucleotide sequence encoded by a human (Tey1) metastasis suppressor gene located at p21-p12 on chromosome 8, or a fragment thereof, wherein the RNA molecule comprises from about 10 to about 100 nucleotides, wherein a decrease in the level of the RNA molecule in the test sample as compared to the level of the RNA molecule in a control sample is diagnostic for the cancer.

27. A method of prognosticating cancer in a mammal, which method comprises:
(a) obtaining a test sample from the mammal, and
(b) assaying the test sample for the level of an RNA molecule comprising a nucleotide sequence encoded by a human (Tey1) metastasis suppressor gene located at p21-p12 on chromosome 8, or a fragment thereof, wherein the RNA molecule comprises from about 10 to about 100 nucleotides, wherein an increase in the level of the RNA molecule over time is indicative of a positive prognosis and a decrease in the level of the RNA molecule over time is indicative of a negative prognosis.

28. The method of claim 27, wherein the method is used to assess the efficacy of treatment of the cancer.


SEQUENCE LISTING (1 sequence)

SEQ ID NO: 1 - DNA, 1014 bp, Homo sapiens
ggggcacgggccatgcctgaaccttcttccaccgccgcgccccgggcccgcgccccgccggccgcccgcactgggtcccgcgtcgcccccgccaccacaccgagccgcccagtacctcctcctcctgaaggcagagctgggggtccgtacggaggaacttcagaggctggaagcacgcttgaaaaagaaagggggcctgggctttgcaccagcttcaggattgcgtacttgtttcctcatccgtgaaatgggagcagctgaatgtgtctcctcgagttttcgagaaaaatgagggtctcactttgttgctcaggctgagctcgagctcctggcctcaagtgatcctcctgcttttggcctcccaaactgatgggattacaacgtggagccaccgtgttaaactcttagatttgactgctacaaaggtataaatgtggaactccatatatcagtgggttctgccccacttcttcctcccaggaattctccatcccagcctgctgctcctgtcttgcaccggggaacaaacatgtatcctggatgaaaataaaccaactcttgaaactgcaacccaagacattcagaagagataaggctacagtgaatgtggaaactttgcacgtgtttggatgtattgattgccttaactggcagctctttgcactgccagcttccctcaattacatgaggaaccacgagggggcaatgtcttcccgagcaataatagctgagtggtggtcttaggaccccgtggtggcccatgatgctagcactgtgtctttggccattcttgcctgtcatggagcatactccacagaagggcatggtcttcttggatcccggcatgggcccaacaaaaatgacaatgtagtcaatagttccactcagacctcacttaggagcaggtgaactatgttgtgtgatatcaggatgcccggagaacctctcctatccccacccagggaagcatggcttaattccaaataaagaatctgatttctttgtctttaagcc